(19) United States
(12) Patent Application Publication
(10) Pub. No.: US 2009/0191294 A1
(43) Pub. Date: Jul. 30, 2009

(54) Grape cane product, and method of making

(76) Inventor: Ahmad Alkayali, Murrieta, CA (US)

Correspondence Address: Law Office of Terry L. Miller, 24832 Via San Fernando, Mission Viejo, CA, 92692, US

(21) Appl. No.: 12/384,052

(22) Filed: Mar. 31, 2009

Related U.S. Application Data
(63) Continuation-in-part of application No. 11/787,512, filed on Apr. 17, 2007.
Application No. 11/787,512 is a continuation-in-part of application No. 12/075,847, filed on Mar. 14, 2008.

(51) Int. Cl. A61K 36/87; A61P 3/02

(52) U.S. Cl. 424/766

(57) ABSTRACT

An extract of the grape plant obtained from grape plant cane is a powerful anti-oxidant, and offers a unique combination of beneficial organic chemicals, including trans-resveratrol, trans-viniferin, and oligostiibines. The grape plant cane product is produced essentially exclusively from grape plant cane, which is often discarded by vintners. Methods of making this product include solvent extraction from the grape plant cane, followed by vacuum drying without overheating the product. The grape plant cane extract may be used as an anti-fungal, antibiotic, antiseptic, preservative, as a topical application to improve moisture retention of human skin, and to extend the shipping and shelf life of produce, among other uses. A method for using the grape plant cane extract as a food supplement involves daily human ingestion of desired amounts of a food-grade grape plant cane product.

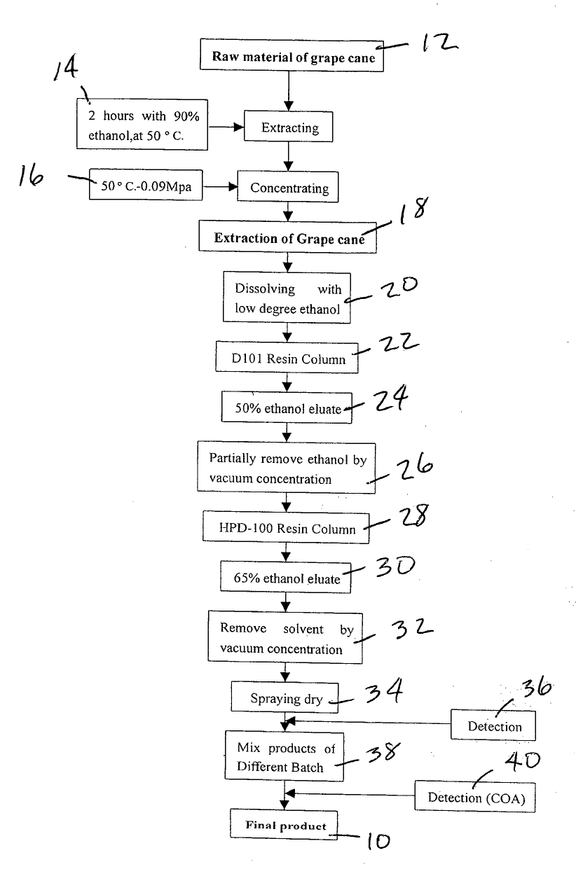

### CROSS REFERENCE TO RELATED APPLICATION

This application is a Continuation-in-Part of U.S. application Ser. No. 11/787,512, filed 17 Apr. 2007, and also is a Continuation-in-Part of U.S. application Ser. No. 12/075,847, filed 14 Mar. 2008, the disclosures of which are incorporated by reference herein to the full extent necessary for a complete and enabling disclosure of the present invention.

### BACKGROUND OF THE INVENTION

### Field of the Invention

This invention relates generally to an extract of grape plant cane containing beneficial or helpful chemical constituents. The grape plant cane extract is believed to have beneficial uses as a food supplement having antioxidant and other healthful effects in the human body. Also, the grape plant cane product is believed to be useful as an antiseptic, anti-fungal, antibiotic, preservative (particularly useful for application to certain fruits and vegetables), as a natural (i.e., organic) pesticide or insect inhibitor; and as an ingredient useful in topical preparations such as face and hand creams, in order to promote preservation of moisture in the human skin. The grape plant cane may include branches, stems, grape skins, grape seeds, leaves, vine trimmings, and other plant parts from various species of grape plants, with some species of grape plant being particularly preferred.

The grape plant cane product according to this invention is made by solvent extraction from the grape cane without the use of elevated temperatures. Thus, oxidation or chemical breaking of the grape plant constituents which might result from such elevated temperatures does not occur in the product made according to this invention. Plant parts from such grape varieties as the red grape plant cane (i.e., stems, branches, and even leaves), or such parts of the white muscadine grape plant, or of other grape plants, or combinations of such plant parts from selected grape plants may be used as the grape cane source materials without limitation

More particularly, this invention relates generally to food supplements containing beneficial or helpful chemical constituents, and particularly relates to a dry powder, granular, or flake type of food supplement which is produced by solvent extraction from natural grape plant parts as a source material. Even more particularly, this invention relates to a powerful anti-oxidant food supplement made from grape cane (i.e., from the branches, stems, leaves, vine trimmings, and other grape plant parts). The product is preferably made by solvent extraction, although the invention is not so limited. For example, the plant source materials may include such parts of the red grape plant cane (i.e., stems, branches, and even leaves), or such parts of the white muscadine grape plant, or of other grape plants, or combinations of such plant parts from selected grape plants without limitation being implied. It is to be noted that especially the seeds of the white muscadine grape plant are particularly favored for inclusion in grape cane to be processed according to the present invention because of their high content of desired constituents in the food supplement product hereby produced. By avoiding the use of elevated temperatures, the food values and nutritional constituents of the grape plant are preserved and are not oxidized or deteriorated by excessively elevated temperatures. Especially important in the context of this present invention is the use of grape plant parts (herein referred to as “grape cane”) which historically has been discarded as trimmings of the grape vines in many vineyards. By use of the grape cane, the yield of product beneficial to humans from the vineyard is increased, and the cost of this product is greatly reduced in comparison to product made exclusively from grapes.

### RELATED TECHNOLOGY

Resveratrol is a phytoalexin produced by several plants and sold as a nutritional supplement. It has also been produced by chemical synthesis. A number of beneficial effects, such as anti-cancer, antiviral, neuroprotective, anti-aging, anti-inflammatory and life-prolonging effects have been reported in non-human species. Resveratrol is found in the skin of red grapes and in a constituent of red wine but apparently not in sufficient amounts to explain the so-called French paradox. That paradox relates to the low incidence of coronary heart disease in southern France despite a diet that is high in saturated fats. It has been found that Resveratrol increases the activity of a protein SIRT1, and that this protein significantly increases the lifespan of yeast and mice. Four stilbenes cis and trans resveratrol, and cis and trans piecid are similar and related, and are sometimes analyzed together as a group. Resveratrol produced by plants apparently has anti-fungal properties, and is found in widely varying amounts in grapes (primarily in the skins) and in the roots and stalks of giant knotweed and Japanese knotweed. Muscadine grapes and muscadine wines are also known to contain a good source of resveratrol. However, no food supplement product is known to the applicant which is rich in desired constituents, and which is made entirely of grape cane.

However, studies have shown that taking resveratrol alone may not be sufficient to obtain the desired protections. According to Dr. Xi Zhao-Wilson, PhD, resveratrol is derived from natural plant extracts, and usually in a mixture with many other molecules. In order to obtain the “optimum bioavailability and synergistic action” of resveratrol, some of the other molecules that make up the stilbenes may be needed. These other molecules may include Trans-e-Viniferin, or (E)-e-Viniferin (a natural phenolic compound—a resveratrol dimer) that, like trans-resveratrol or (E)-resveratrol [3,4′,5-trihydroxy-trans-stibene], is present in grapevine (i.e., in grape cane materials).

Unlike trans-resveratrol, which in vitro, ex vivo, and in vivo experiments has shown a number of biological activities including anti-inflammatory, anti-oxidant, platelet anti-aggregatory, and anti-carcinogenic properties (for reviews, see, e.g., Aggarwal, et al., 2004; Alarcon de la Lastra, and Villegas, 2005; Delmas, et al., 2005; Olas and Wachawicz, 2005; Orallo, 2005; Oallo, 2006; Ulrich, et al., 2005). This newly discovered molecule, e-Viniferin, has been reported to induce apoptosis of leukemia B-cells (Billard, et al., 2002; Quinery, et al., 2004), to inhibit human cytochrome P450 enzymes (Piver, et al., 2003), and to have antioxidant capacity (Privat, et al., 2002).

### SUMMARY OF THE INVENTION

In view of the deficiencies of the conventional technology, an object for this invention is to overcome or ameliorate one or more of those deficiencies.

An object of this invention is to provide an extract of grape plant cane.

An additional object for this invention is to provide a dry product food supplement made exclusively from grape plant cane and containing resveratrol and other beneficial constituents of the grape plant.

Particularly, an object for this invention is to provide a dry product food supplement containing one or more of: Trans-Resveratrol, Trans-e-Viniferin, trans-piecid, trans-astringin, Oligostilbenes, OligomericProanthocyanidin (OPC's), and Polyphenols.

While red wine has long had a recognized benefit as an anti-oxidant, the presence of the beneficial constituents of red wine in the often-discarded grape cane has not been widely recognized. The active ingredient of red wine has increasingly been recognized as resveratrol as well as other beneficial constituents of the red wine. These constituents are also present in grape plant cane, which is employed to make a food supplement product according to one aspect of this invention.

Further to the above, the Applicant has discovered that an extract, or grape plant cane product according to this invention also has beneficial uses as a skin care product, promoting retention of water in the skin tissues.

Also, the grape plant cane product according to this invention has beneficial uses as a preservative, to prolong the shelf life of certain fruits and vegetables while also preserving nutrients and flavor.

Still further, the grape cane product according to this invention may find use as a natural (i.e., organic) pesticide or insect inhibitor.

And finally, a grape cane product according to this invention may be added to other food products, in order both to preserve the freshness and increase shelf life of those products, while also improving the benefits of the products as being healthful for human consumption. To this end, the grape plant cane product according to this invention may be added as an ingredient in, for example, yogurt, chocolate candy, nutrition bars, and cereals.

Particularly, fruits, vegetables, and other agricultural produce are exposed to attack by pathogens, and to natural biological processes of deterioration, aging, spoilage, and the actions of molds and mildews during transportation and storage. The Applicant believes and has evidence to support the use of a grape plant cane extract as a produce preservative.

Resveratrol (3,5,4′-trihydoxystilbene) has been described as one of the most important molecules for resistance to fungal diseases. Moreover, the combination of chemical molecules (i.e., constituents) of the present grape plant cane product have been found to function as a natural antibiotic and anti-fungal agent to reduce microbial and fungal contamination of agricultural products, and to improve their condition after transport and/or storage. Treatment with grape plant cane product according to this invention inhibits mold and yeast development on grapes, tomatoes, mushrooms, and certain other fruits; and preserves water content without adversely affecting fruit firmness or nutrition, for example.

The operation and advantages of the present invention will be better understood by those of ordinary skill in the art and others upon reading the ensuing specification when taken in conjunction with the appended drawing wherein:

### BRIEF DESCRIPTION OF THE DRAWING FIGURE

The single drawing FIGURE depicts a process flow chart for a method of producing a dry product from source materials including grape plant cane.

### DETAILED DESCRIPTION OF THE PREFERRED EMBODIMENT

Turning first to the appended drawing FIGURE, this FIGURE schematically illustrates a process or method for producing a free-flowing dry crystalline or powder (i.e., granular or flake-like) food supplement product 10 using selected source materials (indicated as 12). As is indicated on this FIGURE, the source material 12 is grape plant “cane.” That is, source material 12 includes such grape plant materials as grape plant stems, branches, skins of the grapes, grape seed, and even leaves. Also included in this term “grape cane” is such materials as even the leaf stems and root materials of the grape plant. While the preferred source of these grape plant materials (i.e., the grape plant cane) is the red grape plant, white muscadine grape plants and parts of these grape plants may be included, and especially the seeds of the white muscadine grape. It is important to note that this grape plant cane source material includes many grape plant materials that are simply trimmed off the grape plant vines during maintenance of a vineyard, and which historically were thrown into a land fill or burned. The grape plant cane is most preferably washed to remove debris, and then is ground to a sufficiently small size allowing for efficient solvent extraction, which is further described by reference to the appended drawing FIGURE.

Considering the processing of the grape plant cane source material 12, it is seen on the appended drawing FIGURE that the grape plant cane 12 is treated (i.e., soaked) with an ethanol solution, which is preferably 90 percent ethanol and preferably at approximately 50° C. for a time of about two hours (step 14), although the invention is not so limited. In other words, shorter or lower temperature soaking intervals can be expected to yield less product, while longer soaking yields a modest increase in the product. The Applicant has determined that the indicated soaking interval is a good compromise between processing time requirements and process yield.

The ethanol soaking solution is then drained from the grape plant cane, and this solution of extraction (i.e., from step 14) is concentrated by vacuum concentration or drying. Preferably, this vacuum concentration or drying is carried out at 0.09 Mpa (i.e., partial vacuum), and 50° C. (step 16), although the invention is not so limited. It is to be noted that if the vacuum drying process is extended to produce either a granular or concentrated “liquor” of the grape plant cane at this stage of the process, the manufacturing intermediate produce so produced may have beneficial uses as a food preservative, for example, when sprayed on produce, as is further explained below. It is to be understood that the intermediate product, if produced in a dry form, is dissolved in a suitable solvent for the mentioned intermediate uses.

The resulting semi-dry (possibly as a concentrated “liquor” of the grape plant cane, or dry material of extraction (i.e., from step 16—indicated on the appended drawing FIGURE with the numeral 18) is dissolved with 20 percent ethanol (step 20), and is then passed along a D101 resin column (step 22). The invention is not limited to the use of any particular resin column, but the Applicant has determined that the D101 column works well in the process of making a food grade extract of the grape plant cane.

The captured extraction material in the resin column of step 22 is eluted, preferably using 50 percent ethanol (step 24). Again, the resulting solution, now carrying the desired constituents extracted from the grape plant cane 12 (i.e., placed back into solution at step 24) is again vacuum concentrated or dried at step 26. Preferably, this vacuum concentration is carried out at about 0.09 Mpa, and 50° C. Again, at this stage of the manufacturing process, it is to be noted that if the vacuum drying process is extended to produce either a granular or a concentrated “liquor” of the grape plant cane at this stage of the process, the manufacturing intermediate product so obtained may have beneficial uses as a food preservative, for example, when sprayed on produce, as is further explained below. Again, it is to be understood that the intermediate product, if produced in a dry form, is dissolved in a suitable solvent for the mentioned intermediate uses. Alternatively, the manufacturing intermediate product from step 24 may be used, for example as a fungicide, or as a antiseptic, or in topical products for skin care.

Step 28 illustrates another resin column separation, this time the liquid material from step 26 is preferably passed along a HDP-100 resin column. The captured extraction material in the resin column of step 28 is eluted preferably using 65 percent ethanol (step 30). Again, the resulting solution, now carrying the desired constituents extracted from the grape plant cane 12 (i.e., placed back into solution at step 30) is vacuum concentrated, indicated generally at step 32. Particularly, step 32 includes spray drying (indicated at 34 on the appended drawing FIGURE) at a sufficiently low pressure and low temperature so as to produce a substantially dry, free flowing, powder, granular, or flake-like food-grade product.

At steps 36, 38, and 40 are illustrated optional steps for the food-grade product intended to insure the quality and concentration levels of the finished food supplement product. The first (step 36) includes an assaying step during which the dried material from step 34 is tested for the presence of resveratrol, stilbene, and Viniferin. The levels of these constituents are expected to vary depending on a number of variables, including the type of grape plant cane utilized for a particular batch of product, and the location at which the grape plants are grown, among other variables. Records are kept for successive batches of grape plant cane product. At step 38 is illustrated the mixing or blending of differing batches of grape plant cane product according to the levels of Resveratrol and Viniferin present in these various batches. The desired results of this mixing is to obtain a preferred level of 10% Resveratrol, and 10% Viniferin in each blended batch of food supplement product. The final step (step 40) includes a final assaying of the food supplement product to confirm the results of the blending step (step 38).

The finished food supplement product 10 of step 40 (or step 34) may be packaged for human consumption in the form of a powder, granular material, or flake-like material. The product may be pressed into tablets, or may be placed into capsules. Alternatively, the product 10 may be dissolved in various solvents or carriers for consumption by humans as a liquid or syrup. Moreover, it is to be understood that the final product is suitable as well for the mentioned intermediate-product uses discussed above.

It should be noted that although the temperatures used at all steps of the process illustrated in the appended drawing FIGURE are below the boiling point of water, and vaporization of the extract is accomplished by using sub-ambient (i.e., vacuum) pressures. As a result, the chemical constituents of the finished product are not deteriorated or oxidized by the low temperatures used in the extraction process. In this sense, the product 10 may be considered to be a “cold process” grape plant cane extract.

This product 10 is then packaged (possibly with a grinding step to remove lumps and aggregations) in convenient forms for use as a grape plant food supplement. For example, the dry product may be placed in capsules, may be pressed into pill form, or may be placed loose as a free-flowing dry powder into bottles. Again, alternatively, the product 10 may be dissolved in various solvents, such as water or oils, for consumption as a liquid or syrup. The preferred dosage of the product when used as a food supplement, which may be packaged in capsule, tablet or added to beverages or foods in the dry powder form, is from about 100 mcg to about 500 mg. daily dosage. When this product is utilized as a skin care product for the human shin, which may be presented for use in cream, lotion, or serum base, or as a constituent of a sun screen product, for example, the preferred range of concentration is from about 0.01% to about 5%. Still alternatively, when this product is utilized as a preservative for produce, such as fruits and vegetables, the produce is preferably dipped in a solution of the grape plant extract. The dipping solution is most preferably made with a concentration of about 50 mcg to 100 mg per gallon, and is made of a mixture of 75% distilled water and 25% tap water. The dipping solution is preferably stirred for each treatment of fruit or produce.

I claim:

1. An extract of the grape plant made essentially exclusively from a source material consisting of grape plant cane.

2. The extract of grape plant of claim 1 wherein said extract is utilized as a food supplement, and wherein said grape plant cane source material comprises stems and branches of grape plants.

3. The food supplement of claim 1, wherein said grape plant cane source material is subjected to solvent extraction, and a resulting solution of extraction is dried to provide a dry powder food supplement material.

4. The food supplement of claim 3, wherein the source material, solution of extraction, and food supplement are exposed to processing temperatures always below the boiling point of water at atmospheric pressure.

5. The food supplement of claim 3 packaged in a form for daily human consumption at a daily dose of about 100 mcg to about 500 mg.

6. The food supplement of claim 3, including Trans-Resveratrol, Trans-e-Viniferin, trans-piecid, trans-astringin, Oligostilbenes, OligomericProanthocyanidin, and Polyphenols.

7. The food supplement of claim 6, including substantially 10% Resveratrol, and substantially 10% Viniferin.

8. The grape cane extract of claim 1 wherein said extract is utilized as a human skin care product including from about 0.01% to about 5% of said grape cane extract.

9. The grape cane extract of claim 1 wherein said extract is utilized as a preservative for produce, and said produce is dipped in a solution of water and said grape cane extract at a concentration from about 50 mcg to about 100 mg of grape cane extract.

10. A method of making a grape product food supplement, said method comprising steps of:
providing a first source material including grape plant cane, said grape plant cane including parts of the grape plant vine, such as stems and branches; and
producing from said source materials a dry powder food supplement product which contains beneficial chemical constituents of the grape plant.

11. The method of claim 10 wherein said grape plant cane is first washed and ground, and is then soaked in an ethanol/water mixture at elevated temperature below the boiling point of water at atmospheric pressure for a determined time interval to provide a first solution of extraction.

12. The method of claim 11, further including the step of vacuum concentrating said first solution of extraction.

13. The method of claim 12 further including the step of using ethanol dissolution, resin absorption, and ethanol dissolution, followed by another vacuum concentration to provide a semi-finished product of the grape plant cane source material.

14. A method of making an extract of the grape plant, said method including steps of:
providing a source material including grape plant cane, said grape plant cane including parts of the grape plant vine including stems and branches;
soaking said source material in an ethanol solution;
draining the ethanol solution from the source material as a first solution of extraction;
concentrating said first solution of extraction to provide a concentrated liquor of the grape plant cane;
dissolving or diluting said concentrated liquor of the grape plant cane with 20 percent ethanol to provide a second solution of extraction;
passing said second solution of extraction along a first resin column and capturing in said resin column extracted chemical constituents of said grape plant cane;
eluting captured extracted chemical constituents of the grape plant cane from the resin column to provide a third solution of extraction;
vacuum concentrating the third solution of extraction;
passing said third solution of extraction along a second resin column and capturing extracted chemical constituents of said grape plant cane;
eluted a fourth solution of extraction from said second resin column to provide a forth solution of extraction; and
vacuum concentrating said forth solution of extraction by spray drying said forth solution of extraction at a sufficiently low pressure and low temperature so as to produce a substantially dry, free flowing, powder, granular, or flake-like extract of the grape plant.

15. The method of claim 14 further including the steps of soaking said source material in a solution of substantially 90 percent ethanol at a temperature of substantially 50° C., and for a time of about two hours.

16. The method of claim 14 further including the steps of concentrating said first solution of extraction by vacuum concentration at a vacuum pressure of substantially 0.09 Mpa, and at a temperature of substantially 50° C.

17. The method of claim 14 further including the steps of passing said second solution of extraction along a D101 resin column in order to capture extracted chemical constituents of said grape plant cane.

18. The method of claim 14 further including the steps of eluting the captured extracted chemical constituents of the grape plant cane from the first resin column with a solution of 50 percent ethanol to provide the third solution of extraction.

19. The method of claim 14 further including the steps of vacuum concentrating the third solution of extraction at a vacuum pressure of substantially 0.09 Mpa, and at a temperature so substantially 50° C.

20. The method of claim 14 further including the step of passing said third solution of extraction along an HDP-100 resin column as said second resin column.

21. The method of claim 14 further including the step of eluted the fourth solution of extraction from said second resin column using a solution of substantially 65 percent ethanol to provide the forth solution of extraction.
